(19) United States
(12) Patent Application Publication
(10) Pub. No.: US 2005/0191632 A1
(43) Pub. Date: Sep. 1, 2005

(54) Cytogenetic abnormalities that are predictive of response to therapy for chronic lymphocytic leukemia

(76) Inventors: John Byrd, Columbus, OH (US); Nyla Heerema, Columbus, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/700,264

(22) Filed: Nov. 3, 2003

Related U.S. Application Data
(60) Provisional application No. 60/423,054, filed on Nov. 1, 2002.

(51) Int. Cl. C12Q 1/68; G01N 33/574

(52) U.S. Cl. 435/6; 435/7.23

(57) ABSTRACT

Methods and kits are provided for predicting the response of patients with B-cell chronic lymphocytic leukemia (CLL) to treatment with agents that bind to the CD20 and CD 52 antigens on the surface of B lymphocytes. The methods of the present invention are for identifying patients who are refractory and patients who are responsive to therapy with such agents by analyzing the genome of cells obtained from the patients for the presence of specific chromosomal abnormalities, including del(17p13.1), and one or more of del(13q14.3), del(11q22.3) and trisomy 12. The methods are performed using appropriate cytogenetic analysis techniques, such as fluorescence in situ hybridization (FISH), with probes capable of detecting the specific cytogenetic abnormalities. Patients without del(17p13.1) but with del(13q14.3), del(11q22.3) or trisomy for chromosome 12, have been shown to be responsive to agents that bind CD20, such as rituximab. Patients with del(17p13.1) have been shown not to be responsive to rituximab, but are responsive to agents that bind CD52, such as alemtuzumab. By customizing treatment of CLL based on a patient's cytogenetic profile, an improved Outcome may be achieved for the patient, along with time and cost savings that are afforded by foregoing unnecessary therapy.

### CROSS REFERENCE TO RELATED APPLICATIONS

This application claims the benefit of the filing date of U.S. Provisional Application No. 60/423,054, filed Nov. 1, 2002, which is incorporated herein by reference in its entirety.

This invention is supported, at least in part, by Grant Nos.: CA81534, and P01 CA95426-01A1, from the National Cancer Institute. The U.S. government has certain rights in the invention.

### FIELD OF THE INVENTION

The invention relates to cytogenetic analyses useful for predicting the response of chronic lymphocytic leukemia patients to therapy.

### BACKGROUND

Leukemias are malignant neoplasms of hematopoietic tissues. These neoplasms are categorized into two predominant forms: chronic and acute. While acute leukemias are characterized by undifferentiated cell populations, chronic leukemias usually present a more mature morphology. Notwithstanding these classifications, however, the pathological impairment of normal hematopoiesis is the hallmark of all leukemias.

Chronic lymphocytic leukemia (CLL) is a neoplasm in which a clonal expansion of small lymphocytes accumulates in the marrow, lymph nodes, blood, spleen, liver, and sometimes other organs. The CLL cell is the neoplastic counterpart of an immunologically immature, incompetent lymphocyte. In over 95 percent of the cases, the clonal expansion is of a B-cell lineage. In less than 5 percent of cases the tumor cells have a T-cell phenotype.

While CLL accounts for only about 0.8 percent of all cancers in the United States, it is the most prevalent leukemia afflicting adults in modern countries, and accounts for 30 percent of all leukemias. Ninety percent of CLL patients are over age 50 at the time of disease diagnosis, and the majority are over age 60.

Most patients are diagnosed following a routine physical examination or a blood count. The earliest and most frequent symptoms are fatigue and malaise. Later symptoms include lymphadenopathy and splenomegaly. At diagnosis, anemia and thrombocytopenia are found in approximately 15 percent of patients but become much more frequent as the disease progresses. The disease has a variable natural history with respect to time to progression and response to standard cytotoxic therapies.

Interphase cytogenetic analysis using fluorescence in situ hybridization (FISH) has revealed chromosomal changes in the majority of CLL samples¹⁷ and is superior to standard karyotype analysis for identifying known cytogenetic abnormalites. In the largest comprehensive FISH cytogenetic series published to date⁷, the relative incidence of abnormalities occurring at a frequency of 7% or greater included del(13q14.3), del(11q22-q23), trisomy 12, and del(17p13.1). Several of these abnormalities were shown to have varied clinical significance, with del(13q14) patients having a prolonged time from diagnosis to treatment (median 92 months) and overall survival (median 133 months) while those patients with del(17p13.1) and del(11q22-q23) have a more rapid time from diagnosis to treatment (9 and 13 months) and an inferior survival (32 and 79 months), respectively.⁷

At present, CLL is incurable and existing treatments are for palliating the disease. A variety of drugs exist for this purpose but there have been few studies attempting to correlate possible effectiveness with specific interphase cytogenetic abnormalities of CLL. Four small retrospective series have demonstrated lower response rates to chlorambucil or fludarabine when del(17p13.1) is present.^{2,3,5,6} One preliminary study, including a single case report, demonstrated that CLL patients with del(17p13.1) responded clinically to Campath-1H.¹³ No studies, however, have examined the impact of the more common poor risk interphase cytogenetic abnormality del(11q22-q23). Similarly, there has been little examination of the impact of these abnormalities on therapeutic response to agents, such as agents that are specific for the CD20 antigen or other cell markers, including the monoclonal antibodies rituximab and alemtuzumab.

There is clinical evidence that the presence or absence of one or more cytogenetic abnormalities impact the response to various CLL treatments, which in turn influences patient survival and quality of life. Medical professionals would be better equipped to make treatment decisions regarding CLL if they could understand and predict the likely therapeutic response or resistance of the disease to treatment in a particular patient. Accordingly, there is a need for methods and systems which enable the identification of cytogenetic abnormalities that are predictive of response of CLL cells in a patient to one or more treatments or therapeutic agents. Such methods and systems would permit customization of treatment for CLL based on the particular genetic makeup of each patient.

### SUMMARY OF THE INVENTION

The present invention provides methods and kits for predicting the response of patients with B-cell chronic lymphocytic leukemia (CLL) to treatment with agents that bind to the antigen CD20 on the surface of B lymphocytes. In one embodiment, the methods of the present invention are for identifying patients who are refractory and patients who are responsive to therapy with such agents. One method comprises analyzing the genome of cells obtained from a patient for the presence of del(17p13.1) wherein the presence of this cytogenetic abnormality indicates that the patient will be refractory to treatment with these agents. In a particular embodiment, the method comprises predicting a patient as likely to be refractory to treatment with rituximab when the del(17p13.1) abnormality is detected. Another method comprises analyzing the genome of cells obtained from a patient for the presence of del(17p 13.1), and one or more of del(13q14.3), del(11q22.3) and trisomy 12, the absence of del(17p13.1) and the presence of one or more of the other cytogenetic abnormalities indicating that the patient is likely to respond to treatment with agents that specifically bind to CD20. In yet another embodiment the method comprises predicting a patient as likely to clinically respond to treatment with rituximab where the del(17p13.1) abnormality is absent and one or more of the other cytogenetic abnormalities is present. In a preferred embodiment, the methods are performed using fluorescence in situ hybridization (FISH) with probes capable of detecting the specific cytogenetic abnormalities.

The present invention also provides methods and kits for predicting the response of patients with B-cell chronic lymphocytic leukemia (CLL) to treatment with agents that bind to the antigen CD52 on the surface of B lymphocytes. In one embodiment, the method comprises analyzing the genome of cells obtained from a patient for the presence of del(17p13.1), wherein the presence of this cytogenetic abnormality indicates that the patient will be responsive to treatment with alemtuzumab. In a preferred embodiment, the methods are performed using fluorescence in situ hybridization with probes capable of detecting the specific cytogenetic abnormalities.

Patient cell samples are obtained, for example by drawing blood. Cells, preferably B lymphocytes, are isolated from the blood and are prepared for analysis according to known cytogenetic analysis methods. One such method is fluorescence in situ hybridization (FISH), which is used with specific probes. Probes are used for the purpose of detecting cytogenetic abnormalities, such as del(17p13.1), del(11q22.3), del(13q14.3), and trisomy 12. Examples of probes that can be used in this analysis are available from Vysis (Downers Grove, Ill.) and include: an 145 kb probe called LSI p53 (for 17p13.1); an approximately 500 kb probe that hybridizes to a locus from D11S1828-D11S1294), herein the probe includes a portion that hybridizes with the Ataxia telangiectasia mutated (“ATM”) gene, designated ATM (for 11q22.3); an 130 kb probe called LSI D13S319 (for 13q14.3); and a probe called CEP 12 that is specific for the alpha satellite region at 12p11.1-q11 and detects trisomy 12. The probes are fluorescently labeled and hybridized to polynucleotide targets in cell samples. In one embodiment, after hybridization, the cell samples are viewed under a fluorescence microscope and, after comparison of hybridization of the same probes to control cells that are normal in karyotype, it is determined whether the specific cytogenetic abnormalities are present. Deletions are observed as absence of one or more hybridization signals. Trisomy 12 is observed as presence of an additional signal.

The inventive methods are advantageous in that they indicate whether treatment of a specific patient with a therapeutic regimen, such as with agents specific for the CD20 antigen, for example rituximab, or with agents such as alemtuzumab, which is specific for the CD52 antigen, will or will not be therapeutically beneficial. For example, patients without del(17p13.1) but with del(13q14.3), del(11q22.3) or trisomy for chromosome 12, have been shown to be responsive to rituximab, thus, the drug can be administered to such patients with knowledge that it will be effective. On the other hand, patients with del(17p13.1) have been shown not to be responsive to rituximab but are responsive to alemtuzumab. By customizing treatment of CLL based on a patient's cytogenetic profile, an improved outcome may be achieved for the patient, along with time and cost savings that are afforded by foregoing unnecessary therapy.

### DETAILED DESCRIPTION OF THE INVENTION

Patients

The method of testing patients to determine sensitivity or resistance to rituximab therapy is used on cell samples from individuals who have been diagnosed with CLL or on cell samples from individuals who have not yet been diagnosed with CLL but show symptoms or have a predisposition to having CLL. Individuals who have already been diagnosed as having CLL may or may not have already received one or more therapeutic treatments for CLL.

Diagnosis of CLL is well-known in the practice of hematology. Individuals with CLL often present with an incidental finding of lymphocytosis (increase in lymphocyte number). Results of physical examination, at least initially, may be normal or may reveal minimal, diffuse, nontender adenopathy or splenomegaly. Common complaints are fatigue, malaise and occasional fever or night sweating. In more advanced stages, patients may have weight loss and neck masses. As the disease progresses, the white blood cell count increases over time and anemia, thrombocytopenia and recurrent infectious diseases frequently develop. The progress of the disease in a patient can be determined using various classification schemes. Two such classification schemes are those of Rai et al. (Blood 46:219, 1975) and Binet et al. (Cancer 40:855, 1981). The Rai system classifies a patient as in stages 0 (least advanced disease) through IV (advanced disease).

Patient Cell Samples

Since CLL is a disease that affects predominantly B cells, these are the cells that are obtained from the blood or bone marrow of patients to be tested by an appropriate cytogenetic testing method known in the art, for example, the FISH method. Lymphocytes are obtained by methods well known in the art. Such methods can include gradient centrifugation of whole blood or bone marrow aspirate. Blood can also be cultured with B-cell mitogens prior to lymphocyte isolation and testing. In one method, blood drawn from patients is centrifuged at low speed to obtain buffy coats, which contain white blood cells. Buffy coats are then subjected to density gradient centrifugation (e.g., ficoll-hypaque) to enrich for lymphocytes.

Cell Preparation

The cells are washed, and resuspended in biological buffer. The cells are then treated in hypotonic solution (e.g., 0.075 M KCl). Hypotonic pretreatment induces swelling of the cells and bursts open any contaminating red blood cells. The cells are then fixed. Fixation typically uses, for example, a 3:1 methanol:acetic acid solution. Other fixatives can be acid alcohol solutions, acid acetone solutions, or aldehydes such as formaldehyde, paraformaldehyde, and glutaraldehyde. Fixation helps keep the cells in a “swollen” state, achieved after hypotonic treatment. The fixative solution makes the cell membrane more fragile and suitable for spreading flat on the slide when subjected to the drying techniques.

Cells are placed on slides. Commercially pre-cleaned slides give good results. Cells are dropped onto slides and allowed to dry. The cell suspension is applied to slides such that the cells do not overlap on the slide. Cell density can be measured by microscopy.

Optionally, for use according to the FISH method, the slides are then aged. In FISH the purpose of aging is (1) to fix the biologic material to the glass surface and (2) to increase the “hardness” of the chromosomes, making their structure resistant to the subsequent DNA denaturing. At least two different approaches can be used for aging—dry heat and chemical aging.

Prior to in situ hybridization, chromosomal DNA contained within the cells is denatured. Denaturation typically is performed by incubating in the presence of heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3M sodium chloride and 0.03 M sodium citrate). Denaturation conditions typically are established such that cell morphology is preserved (e.g., relatively low temperatures and high formamide concentrations).

Probes to Cytogenetic Abnormalities

The probes contain DNA segments that are essentially complementary to DNA base sequences existing in different portions of the chromosomes. Examples of probes useful according to the invention, and labeling and hybridization of probes to samples are described in two U.S. patents to Vysis, Inc. U.S. Pat. Nos. 5,491,224 and 6,277,569 to Bittner, et al.

Chromosomal probes are typically about 50 to about 10⁵ nucleotides in length. Longer probes typically comprise smaller fragments of about 100 to about 500 nucleotides in length. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, Biotechnic Histochem., 1998, 73(1):6-22, Wheeless et al., Cytometry 1994, 17:319-326, and U.S. Pat. No. 5,491,224.

The probes to be used hybridize to a specific region of a chromosome to determine whether a cytogenetic abnormality is present in this region. One type of cytogenetic abnormality is a deletion. Although deletions can be of one or more entire chromosomes, deletions normally involve loss of part of one or more chromosomes. If the entire region of a chromosome that is contained in a probe is deleted from a cell, hybridization of that probe to the DNA from the cell will normally not occur and no signal will be present on that chromosome. If the region of a chromosome that is partially contained within a probe is deleted from a cell, hybridization of that probe to the DNA from the cell may still occur, but less of a signal may be present. Preferably, the loss of a signal is compared to probe hybridization to DNA from control cells that do not contain the genetic abnormalities which the probes are intended to detect. Preferably, 200 cells are enumerated for presence of the cytogenetic abnormality.

Another type of cytogenetic abnormality is a duplication. Duplications can be of entire chromosomes, or of regions smaller than an entire chromosome. If the region of a chromosome that is contained in a probe is duplicated in a cell, hybridization of that probe to the DNA from the cell will normally produce at least one additional signal as compared to the number of signals present in control cells with no abnormality of the chromosomal region contained in the probe. Although any probes that detect del(17p13.1) or del(13q14.3) or del(11q22.3) or trisomy for chromosome 12 can be used, suitable probes are available from Vysis, Inc. (Downers Grove, Ill.).

One probe is the LSI p53 probe for 17p13.1. This probe is approximately 145 kb in length and maps to the 17p13.1 region on chromosome 17 containing the p53 gene. This probe may be used to detect the deletion (not mutation) or amplification of the p53 locus.

Another probe is the LSI D13S319 probe for 13q14.3. This probe is approximately 130 kb in length. This probe may be used to identify deletions of the LSI D13S319 locus at 13q14.3. The LSI D13S319 region is located between RB1 and the D13S25 loci. A candidate tumor suppressor gene resides telomeric of the RB1 gene at 13q14.

Another probe is the CEP 12 probe for centromere 12. This probe is specific for the alpha satellite region at 12p11.1-q11. The CEP 12 DNA probe hybridizes to the alpha satellite (centromeric) region (12p11.1-q11) of chromosome 12.

The Vysis LSI and CEP probes are described in U.S. Pat. No. 5,491,224 to Bittner et. al., which is herein incorporated by reference in its entirety.

Another probe, designated ATM, is for 11q22.3 and is approximately 500 kb. This probe hybridizes to a locus from D11S1828-D11S1294 including ATM. This probe is available from Vysis, Inc.

Although the above described probes are preferred, other probes can be used in practice of the invention, as long as the probes are specific for the regions of human chromosomes indicated and as long as they detect the specific cytogenetic abnormalities indicated.

Probe Preparation

Chromosomal probes are labeled so that the chromosomal region to which they hybridize can be detected. Probes typically are directly labeled with a fluorophore, an organic molecule that fluoresces after absorbing light of lower wavelength/higher energy. The fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, U.S. Pat. No. 5,491,224.

The U.S. Pat. No. 5,491,224 describes probe labeling as a number of the cytosine residues having a fluorescent label covalently bonded thereto. The number of fluorescently labeled cytosine bases is sufficient to generate a detectable fluorescent signal while the individual so labeled DNA segments essentially retain their specific complementary binding (hybridizing) properties with respect to the chromosome or chromosome region to be detected. Such probes are made by taking the unlabeled DNA probe segment, transaminating with a linking group a number of deoxycytidine nucleotides in the segment, covalently bonding a fluorescent label to at least a portion of the transaminated deoxycytidine bases.

Probes can also be labeled by nick translation, random primer labeling or PCR labeling, Labeling is done using either fluorescent (direct)-or haptene (indirect)-labeled nucleotides. Some possible labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP or Dinitrophenyl (DNP)-11-dUTP.

Probes also can be indirectly labeled with biotin or digoxygenin, or labeled with radioactive isotopes such as ³²p and ³H, although secondary detection molecules or further processing then is required to visualize the probes. For example, a probe labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Probes can also be prepared such that a fluorescent or other label is not part of the DNA before or during the hybridization, and is added after hybridization to detect the probe hybridized to a chromosome. For example, probes can be used that have antigenic molecules incorporated into the DNA. After hybridization, these antigenic molecules are detected using specific antibodies reactive with the antigenic molecules. Such antibodies can themselves incorporate a fluorochrome, or can be detected using a second antibody with a bound fluorochrome.

However treated or modified, the probe DNA is commonly purified in order to remove unreacted, residual products (e.g., fluorochrome molecules not incorporated into the DNA) before use in hybridization.

Before use in hybridization, chromosomal probes are denatured. Denaturation is performed by heating. For example, probes can be heated to about 73° C. for about five minutes.

Hybridization

In general, the steps comprise adding an excess of blocking DNA to the labeled probe composition, contacting the blocked probe composition under hybridizing conditions with the chromosome region to be detected, preferably on a slide where the DNA has been denatured, washing away unhybridized probe, and detecting the binding of the probe composition to the chromosome or chromosomal region.

Probes are hybridized or annealed to the chromosomal DNA under hybridizing conditions. “Hybridizing conditions” are conditions that facilitate annealing between a probe and target chromosomal DNA. Since annealing of different probes will vary depending on probe length, base concentration and the like, annealing is facilitated by varying probe concentration, hybridization temperature, salt concentration and other factors well known in the art.

Hybridization conditions are facilitated by varying the concentrations, base compositions, complexities, and lengths of the probes, as well as salt concentrations, temperatures, and length of incubation. For example, in situ hybridizations are typically performed in hybridization buffer containing 1-2×SSC, 50-65% formamide and blocking DNA to suppress non-specific hybridization. In general, hybridization conditions, as described above, include temperatures of about 25° C. to about 55° C., and incubation lengths of about 0.5 hours to about 96 hours.

Non-specific binding of chromosomal probes to DNA outside of the target region can be removed by a series of washes. Temperature and concentration of salt in each wash are varied to control stringency of the washes. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

After washing, the slide is allowed to drain and air dry, then mounting medium, a counterstain such as DAPI, and a coverslip are applied to the slide. Slides can be viewed immediately or stored at −20° C. before examination.

Probes are viewed with a fluorescence microscope equipped with an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the chromosomal probes.

FISH

Fluorescence in situ hybridization (FISH) uses fluorescent molecules to identify genes or chromosomes. FISH involves the preparation of short sequences of single-stranded DNA (probes), complementary to the DNA sequences the researchers wish to identify and examine. For example, FISH can be used to detect chromosome copy number or rearrangement of regions of chromosomes. These probes hybridize, or bind, to the complementary DNA and, because they are labeled with fluorescent tags, allow researchers to see the location of those sequences of DNA using a fluorescence microscope. Unlike most other techniques used to study chromosomes, which require that the cells be actively dividing, FISH can also be performed on non-dividing cells, making it a highly versatile procedure. Therefore, FISH can be performed using interphase cells, or cells in metaphase of the cell division cycle. Many of the techniques involved in FISH analysis are described in U.S. Pat. No. 5,447,841 by Gray and Pinkel.

Interpreting FISH Results

Normally, hybridization of a probe to chromosomes from cells in FISH uses control cells that are known not to contain the specific cytogenetic abnormality the probe is designed to detect. The FISH hybridization pattern of the probe to DNA from the control cells is compared to hybridization of the same probe to the DNA from cells that are being tested or assayed for the specific cytogenetic abnormality.

When a probe is designed to detect a deletion of a chromosome or chromosomal region, there normally is less hybridization of the probe to DNA from the cells being tested than from the control cells. Normally, there is absence of a probe signal in the tested cells, indicative of loss of the region of a chromosome the probe normally hybridizes to.

When a probe is designed to detect a chromosomal duplication or addition, there normally is more hybridization of the probe to DNA from the cells being tested than from the control cells. Normally, there is addition of a probe signal in the tested cells, indicative of the presence of an additional chromosomal region that the probe normally hybridizes to.

Preferably, two hundred cells are examined for signal and evaluated for the abnormality based on predetermined control limits. Automated instruments are available for counting cell signals that are then reviewed by a trained analyst.

Therapeutic Agents that are Specific for the CD20 Antigen

Rituximab is a chimeric murine/human monoclonal antibody based on human immunoglobulin G (IgG). It binds to the antigen CD20. CD20 antigen is found in normal and malignant pre-B and mature B lymphocytes, including those in over 90% of B-cell non-Hodgkin's lymphomas (NHL) (“CD20+ lymphocytes”). The antigen is absent in hematopoietic stem cells, activated B lymphocytes (plasma cells) and normal tissues (“CD20− lymphocytes”). Rituximab causes lysis of the B lymphocytes by binding to and thereby activating the complement cascade and immune effector cells (antibody-dependent cell-mediated cytotoxicity), and inducing apoptosis. Rituximab depletes (eliminates) B lymphocytes, including malignant B-cells, from peripheral blood, lymph nodes and bone marrow, but does not affect hematopoietic stem cells.

Therapeutic Agents that are Specific for the CD52 Antigen

Alemtuzumab is a humanized rat monoclonal antibody based on human immunoglobulin G (IgG). It binds to the antigen CD52. CD52 antigen is found in normal and malignant B and T lymphocytes, as well as other cells of the immune system, and of the male reproductive system (“CD52+ lymphocytes”). Alemtuzumab causes lysis of the lymphocytes by binding to and thereby activating the complement cascade and immune effector cells (antibody-dependent cell-mediated cytotoxicity), and inducing apoptosis. Alemtuzumab depletes (eliminates) B lymphocytes, including malignant B-cells, from peripheral blood, lymph nodes and bone marrow; it also affects other blood cells, often necessitating the provision of blood transfusions to patients receiving alemtuzumab.

Methods of Predicting Response to Therapeutic Agents

In one aspect, the present invention provides methods for predicting the response of chronic lymphocytic leukemia cells in a patient to treatment with a therapeutic agent that specifically binds to CD20 on CD20+ B lymphocytes. More particularly, the methods involve cytogenetic screening of biological tissue sample from a patient who has been diagnosed with or is suspected of having CLL (i.e., presents with symptoms of CLL). The specific cytogenetic abnormalities that are screened include preferably del(17p13.1), and optionally, one or more of del(13q14.3), del(11q22-q23) and trisomy 12. The results of the screening method and the interpretation thereof are predictive of the patient's response to treatment with CLL therapeutic agents that bind to the antigen CD20 on the surface of B lymphocytes, and cause lysis and apoptosis of the B lymphocytes by activating the complement cascade and immune effector cells, thereby depleting B lymphocytes from peripheral blood, lymph nodes and bone marrow. In one embodiment, the methods are useful for predicting the response of a patient to treatment with rituximab. According to the present invention, the presence of the del(17p13.1) abnormality is indicative that treatment with rituximab will be ineffective against the CLL cells. The absence of the del(17p13.1) abnormality and the presence of one or more of del(13q14.3), del(11q22-q23) and trisomy 12 abnormalities indicates that treatment with rituximab will be at least partially effective.

In another aspect, the present invention provides methods for predicting the response of chronic lymphocytic leukemia cells in a patient to treatment with a therapeutic agent that specifically binds to CD52 on CD52+ B lymphocytes. More particularly, the methods involve cytogenetic screening of biological tissue sample from a patient who has been diagnosed with or is suspected of having CLL. The specific cytogenetic abnormality that is screened is the mutation of the p53 gene, using a probe that is specific for this mutation on chromosome 17, del(17p13.1). The results of the screening method are predictive of the patient's response to treatment with CLL therapeutic agents that bind to the antigen CD52 on the surface of B lymphocytes, and cause lysis and apoptosis of the B lymphocytes by activating the complement cascade and immune effector cells, thereby depleting B lymphocytes from peripheral blood, lymph nodes and bone marrow. In one embodiment, the methods are useful for predicting the response of a patient to treatment with alemtuzumab.

According to the present invention, the presence of the del(17p13.1) abnormality is indicative that treatment with alemtuzumab will be at least partially effective.

A variety of methods and techniques that are well known in the art may be used for the screening analysis, including metaphase cytogenetic analysis by standard karyotype methods, FISH, spectral karyotyping or MFISH, and comparative genomic hybridization.

In one embodiment, the methods of the present invention comprise contacting a DNA sample, preferably a genomic DNA sample, more preferably a chromosomal sample, obtained from cells isolated from the patient to polynucleotide probes that are specific for and hybridize under stringent conditions with genomic DNA in chromosomal regions associated with cytogenetic abnormalities to determine the presence or absence of one or more of the abnormalities in the cells of the patient. The results of the analysis are predictive of the patient's likely response to treatment with therapeutic agents, particularly agents that bind to either the CD52 antigen or the CD20 antigen on the surface of B lymphocytes and cause lysis and apoptosis of the B lymphocytes by activating the complement cascade and immune effector cells, thereby depleting B lymphocytes from peripheral blood, lymph nodes and bone marrow, more particularly the therapeutic agents rituximab and alemtuzumab.

Specific examples of probes that may be used according to the present invention, particularly in FISH analysis, include the DNA probes: LSI p53, which targets the 17p13.1 region of chromosome 17; LSI D13S319, which targets the 13q14.3 region of chromosome 13; CEP 12, which targets the 12p11.1-q11 region of chromosome 12, and will detect trisomy 12; and ATM, which targets the 11q22.3 region of chromosome 11.

### EXAMPLES

Further details of the invention can be found in the following examples, which further define the scope of the invention. All references cited herein are expressly incorporated by reference herein in their entirety.

### Example 1

### Patient Samples and Cell Processing

The patients represent 31 consecutive patients with CLL as defined by the modified NCI 96 criteria⁸ who received thrice weekly rituximab as previously described⁹ for whom pre-treatment cryopreserved samples were available for interphase cytogenetic analysis. Written informed consent was obtained from all patients prior to procurement of cells. Response was judged at 2 months post-therapy according to the modified NCI criteria.⁸ CLL cells were obtained prior to rituximab treatment and mononuclear cells were isolated from peripheral blood using density-gradient centrifugation (Ficoll-Paque Plus, Pharmacia Biotech, Piscataway, N.J.). The cells were then viably cryopreserved in 10% DMSO, 40% fetal calf serum and 50% RPMI media.

### Example 2

### Fluorescence In Situ Hybridization

Cells from 31 CLL patients were thawed rapidly, washed twice in phosphate buffered saline (PBS), diluted to 1×10⁶ cells/ml and treated with 0.075 M KCl for 15 minutes at 37°. The cells were fixed in 3:1 methanol:acetic acid and slides for FISH were made and hybridized with probes for del(17p13.1), del(13q14.3), del(11q22.3), and centromere 12. These probes are commercially available from Vysis, Inc. The LSI p53 (17p13.1) is 145 kb; LSI D13S319 (13q14.3) is approximately 130 kb; CEP 12 for centromere 12 probes the alpha satellite region at 12p11.1-q11. The fourth probe, ATM, for 11q22.3, is approximately 500 kb and hybridizes to a locus from D11S1828-D11S1294 including ATM. All are labeled in SpectrumOrange™ (Vysis, Inc.), and some are also available labeled in SpectrumGreen™ (Vysis, Inc.). The slides were viewed using a Zeiss Axioskop fluorescence microscope equipped with the appropriate filters and imaging software (Perspective System Instrumentation). The number of signals was evaluated in 200 cells for each probe. Standard quality control procedures were used. A control sample was run concurrently with each test run. Prior to testing patient samples, appropriate specificity and sensitivity were established as specified¹⁰ on cells isolated and cryopreserved in a similar manner as described for the CLL cells above. The mean+3 standard deviations, considered positive for a cytogenetic abnormality in these CLL samples were 4% for centromere 12, 10% del(13q14.3), 9% del(17p13.1), and 10% del(11q22.3). Comparisons of response by abnormalities used Fisher's exact test with two-sided p-values performed with SPSS version 11.0 statistical software.

A total of 31 consecutive CLL patients for whom viably preserved CLL cells were available prior to therapy were studied for the presence of the four most common interphase cytogenetic abnormalities. Of these 31 patient samples, successful hybridization of all the probes was possible in 28 (90%). The clinical characteristics of these 28 patients include a median age of 64 with 7 (25%) being female. The patients received a median of 3 (range 0-6) therapies, and 15 (54%) were fludarabine refractory. Advanced stage (modified Rai 3 or 4) was present in 20 (71%) patients.

Interphase cytogenetic abnormalities were noted in 25 of the 28 patients with adequate FISH samples. The frequency of abnormalities noted were del(13q14.3) [n=16, 57%], del(11q22.3) [n=10, 36%], +12 [n=6, 21%], and del(17p13.1) [n=5, 18%]. Three patients [11%] lacked any of these genetic lesions. The del(13q14.3) was monoallelic loss in 15 patients and biallelic in the remaining patient. Interphase abnormalities were noted in isolation in 7 (44%) patients with del(13q14.3) abnormality, 3 (33%) with del(11q22.3), 3 (50%) with trisomy 12, (30%), and 1 (20%) with del(17p13.1). A hierarchical classification was utilized to stratify outcome as previously described⁷ included 5 patients with del(17p13.1), 9 with del(11q22.3), 7 with del(13q14.3), 3 with trisomy 12 and 3 patients with no FISH abnormality.

### Example 3

### Hierarchical Classification System

As described above (Example 2), because the CLL samples often were found to have greater than one detectable cytogenetic abnormality, a hierarchical model of genetic subgroups was used to allocate the samples with multiple abnormalities to one category only. This allocation has been described previously⁷ and is described below.

The five major categories are defined as follows: i) patients with a 17p deletion, ii) patients with an 11 q deletion but not a 17p deletion, iii) patients with 12q trisomy but not a 17p or 11q deletion, iv) patients with a 13q deletion as the only abnormality, and v) patients with normal copy number of these probes.

### Example 4

### Treatment of Patients with Rituximab

Rituximab was administered on a thrice weekly dosing schedule, for four weeks, as previously described.⁹ Specifically, the treatment schedule is described below.

Before each of the 12 treatments, diphenhydramine (50 mg intraveneously (IV)) and acetaminophen (650 mg orally) were administered. For the first treatment, a 100 mg dose (regardless of weight/body surface area) of rituximab was administered over 4 hours (25 mg/h) without dose escalation. If rigors were noted, rituximab administration was ceased temporarily and meperidine 25 mg IV and promethazine 12.5 mg IV (if needed) were administered. If transient bronchospasm was noted, rituximab administration was ceased and the patient was treated with hydrocortisone 100 mg IV and an albuterol (or other B₂ agonist) inhaler. Other infusion-related side effects (dyspnea, hypoxemia, and hypotension) resulted in temporary cessation of the rituximab infusion and were followed by appropriate medical intervention. When these had resolved to grade 1 or less in severity, rituximab administration was reinitiated at half the previous rate.

In addition to the above, for some patients, infusions 2-12 were administered on t three times per week schedule for 4 weeks. Patients received a full dose of either 250 mg/m2 or 375 mg/m2. These rituximab treatments were administered at an initial rate of 50 mg/h, and increased by 100 mg/h increments at 30-minute intervals, to a maximum of 400 mg/h.

Other patients received the first two administrations of rituximab as described above. Then, beginning with the third administration of the drug, rituximab was initiated at an initial rate of 50 mg/h for 15 minutes, and then increased to a rate to ensure the entire dose of the drug was administered over a 1 hour period.

### Example 5

### Response of Patients to Rituximab

Comparisons of response by abnormalities used Fisher's exact test with two-sided p-values performed with SPSS version 11.0 statistical software.

Partial responders are as previously defined.⁸ According to the published standards, to be considered a partial responder, the patient must display #1 below and #2 and/or #3 (if abnormal prior to therapy), as well as one or more of #3, #4 and #6 for at least 2 months. In addition, the presence or absence of constitutional symptoms were recorded.

#1. ≧50% decrease in peripheral blood lymphocyte count from the pretreatment baseline value.

#2. ≧50% reduction in lymphadenopathy.

#3. ≧50% reduction in the size of the liver and/or spleen.

#4. Polymorphonuclear leukocytes ≧1,500/μL or 50% improvement over baseline.

#5. Platelets >100,000 μL or 50% improvement over baseline.

#6. Hemoglobin >11.0 g/dL or 50% improvement over baseline without transfusions.

The response to rituximab for these patients utilizing the cytogenetic hierarchical classification is shown in Table 1.

TABLE 1
Response to Rituximab Therapy by Prioritized
Interphase Cytogenetics

| | | | | No. (%) with |
|---|---|---|---|---|
| | | No (%) Rai | | Partial |
| | Total | Stage | No. (%) Flu | Response to |
| | Patients | III or IV | Refractory | Rituximab |
| del(13q14.3) | 7 | 6 (86) | 4 (57) | 6 (86) |
| del(11q22.3) | 9 | 7 (78) | 6 (66) | 6 (66) |
| del(17p13.1) | 5 | 3 (60) | 2 (40) | 0 (0) |
| +12 | 4 | 1 (25) | 2 (50) | 1 (25) |
| Normal | 3 | 3 (100) | 1 (33) | 0 (0) |
| Key: No-number, Flu-fludarabine, Prioritization assigned based upon presence of specific interphase cytogenetic abnormality | | | | |
| # in descending order as outlined: del(17p13.1) > del(11q22.3) > +12 > del(13q14.3). | | | | |

Response to rituximab varied significantly based upon the prioritized cytogenetic abnormality as shown in Table 1. None of the five patients with del(17p13.1) responded while responses were noted in 12 of the 23 (52%) patients without this abnormality (p=0.05). Only two of these del(17p 13.1) patients were fludarabine refractory at the time of rituximab treatment. While patients with del(11q22.3) have been noted to have rapid progression and inferior survival, 6 of the 9 (66%) patients with this abnormality responded to rituximab. The overall difference in response among the del(11q22.3) and the del(17p13.1) patients was significantly different (p=0.03). Similarly, 6 of the 7 (86%) patients with the del(13q14.3) abnormality responded to rituximab therapy which was significantly higher (p=0.02) than that observed with del(17p13.1).

### REFERENCES

1. Jarosova M, Jedlickova K, Holzerova M, Urbanova R, Papajik T, Raida L, Pikalova Z, Lakoma I, Prekopova I, Kropackova J, Indrak K. Contribution of comparative genomic hybridization and fluorescence in situ hybridization to the detection of chromosomal abnormalities in B-cell chronic lymphocytic leukemia. Onkologie, 24:60-5, 2001.
2. Callet-Bauchu E, Salles G, Gazzo S, Poncet C, Morel D, Pages J, Coiffier B, Cocul P. Felman P. Translocations involving the short arm of chromosome 17 in chronic B-lymphoid disorders: frequent occurrence of dicentric rearrangements and possible association with adverse outcome. Leukemia, 13: 460-468, 1999.
3. Cano I, Martinez J, Quevedo E, Pinilla J, Martin-Recio A, Rodriguez A, Castaneda A, Lopez R, Perez-Pino T, Hemandez-Navarro F. Trisomy 12 and p53 deletion in chronic lymphocytic leukemia detected by fluorescence in situ hybridization: association with morphology and resistance to conventional chemotherapy. Cancer Genet Cytogenet, 90:118-24, 1996.
4. Dohner H, Stilgenbauer S, James M R, Benner A, Weilguni T, Bentz M, Fischer K, Hunstein W, Lichter P. 11q deletions identify a new subset of b-cell chronic lymphocytic leukemia characterized by extensive nodal involvement and inferior prognosis. Blood, 89: 2516-2522, 1997.
5. Chevallier P, Penther D, Avet-Loiseau H, Robillard N, Ifrah N, Mahe B, Hamidou M. Maisonneuve H, Moreau P, Jardel H, Harousseau J L, Bataille R, Garand R CD38 expression and secondary 17p deletion are important prognostic factors in chronic lymphocytic leukaemia. Br J Haematol, 116:142-5, 2002.
6. Dohner H, Fischer K, Bentz M, Hansen K, Benner A, Cabot G, Diehl D, Schlenk R, Coy J, Stilgenbauer S, Lichter P. p53 gene deletion predicts for poor survival and non-response to therapy with purine analogs in chronic B-cell leukemias. Blood, 85:1580-9, 1995.
7. Dohner H, Stilgenbauer S, Benner A, Leupolt E, Krober A, Bullinger L, Dohner K, Bentz M, Lichter P. Genomic abnormalities and survival in chronic lymphocytic leukemia. N Eng J Med, 343: 1910-1916, 2000.
8. Cheson B D, Bennett J M, Grever M, Kay N, Keating M J, O'Brien S, Rai K R National Cancer Institute-sponsored working group guidelines for chronic lymphocytic leukemia: Revised guidelines for diagnosis and treatment. Blood, 87: 4990-4997, 1996.
9. Byrd J C, Murphy T, Howard R S, Lucas M S, Goodrich A, Park K, Pearson M, Waselenko J K, Ling G, Grever M R, Grillo-Lopez A J, Rosenberg J, Kunkel L, Flinn I W. Rituximab using a thrice weekly dosing schedule in B-cell chronic lymphocytic leukemia and small lymphocytic lymphoma demonstrates clinical activity and acceptable toxicity. J Clin Oncol, 19:2153-64, 2001.
10. American College of Medical Genetics, Standards and Guidelines for Clinical Genetics Laboratories, 2^{nd} Ed, Bethesda, Md., 1999.
11. Pedersen I M, Buhl A M, Klausen P, Geisler C H, Jurlander J. The chimeric anti-CD20 antibody rituximab induces apoptosis in B-cell chronic lymphocytic leukemia cells through a p38 mitogen activated protein-kinase-dependent mechanism. Blood, 99:1314-9, 2002.
12. Byrd J C, Kitada S, Flinn I W, Aron J L, Pearson M, Lucas D, Reed J C. The mechanism of tumor cell clearance by rituximab in vivo in patients with B-cell chronic lymphocytic leukemia: evidence of caspase activation and apoptosis induction. Blood, 99:1038-43, 2002.
13. Stilgenbauer S, Scherer K, Krober A, Bullinger L, Hochsmann B, Mayer-Steinacker R, Bunjes D, Dohner H. Campath-1H in refractory B-CLL-Complete Remission Despite p53 gene mutation. Blood, 98: 771a, 2001.
14. Byrd J C, Peterson B L, Morrison B L, Park K, Jacobson R, Hoke E, Rai K, Schiffer C A. Larson R A. Randomized Phase II Study of Fludarabine with Concurrent Versus Sequential Treatment with Rituximab in Symptomatic, Untreated Patients with B-Cell Chronic Lymphocytic Leukemia: Results from CALGB 9712. Blood, 2002 (in press).
   Wierda W, O'Brien S, Albitar M, Lerner S, Plunkett W, Giles F, Andreeff M, Cortes J, Faderl S, Thomas D, Koller C, Kantarjian H, Keating M. Combined fludarabine, cyclophosphamide, and rituximab achieves a high complete remission rate as initial treatment of chronic lymphocytic leukemia. Blood, 98: 721a, 2001 (abstr).

### Example 6

### Treatment of CLL Patients with Alemtuzumab

Chronic lymphocytic leukemia (CLL) is one the most common type of leukemia observed in the Western Hemisphere. While the natural history of CLL is quite varied, patients with p53 gene deletions [del(17p13.1] or p53 point mutations become symptomatic soon after diagnosis and have an inferior survival. The impact of this abnormality on treatment is quite relevant, as several studies have demonstrated that chlorambucil, fludarabine, and rituximab therapy is ineffective in patients who have del(17)(p13.1).¹⁻⁴ Identifying therapies that are effective against this genetic subtype of CLL therefore would represent a major advance for the treatment of CLL.

Alemtuzumab is a humanized anti-CD52 monoclonal antibody that recently was approved by for clinical use in fludarabine-refractory CLL where an overall response rate of 33% was noted.⁵ No molecular studies were performed as part of this trial or others performed with alemuzumab to ascertain its effectiveness in CLL with p53 mutations and/or deletions. Only one case report has noted alemtuzumab might be effective in CLL with p53 mutations and/or deletions.⁶ Herein, we examine a large series of alemtuzumab treated patients and demonstrate clinical activity.

Patient Samples and Cell Processing: The patients represent 36 consecutive patients with CLL as defined by the modified NCI 96 criteria⁷ who received alemtuzumab at our institutions as prescribed for whom pre-treatment cryo-preserved samples were available for assessment of p53 mutation and/or deletions. Written informed consent was obtained from all patients prior to procurement of cells. Patients were assessed with a detailed clinical evaluation (physical exam with lymph node, liver, and spleen measurement; and CBC with differential) two months after completing therapy. For patients attaining a clinical CR, a bone marrow biopsy and aspirate was also performed at these times. Criteria for response utilized the Revised 1996 NCI-sponsored Working Group Guidelines.⁷ As specified by these guidelines, a response had to be maintained for a period of 2 months. CLL cells were obtained prior to alemtuzumab treatment and mononuclear cells were isolated from peripheral blood using density-gradient centrifugation (Ficoll-Paque Plus, Pharmacia Biotech, Piscataway, N.J.). The cells were then viably cryopreserved in 10% DMSO, 40% fetal calf serum and 50% RPMI media.

Fluorescence in situ hybridization and p53 mutational studies: Cells from 36 CLL patients were thawed rapidly and examined for the presence of del(17p13.1) as previously reported by our group using the Vysis, Inc. LSI p53 (17)(p13.1) probe.⁴ Mutations of the p53 gene were assessed by extracting DNA using the QlAamp kit according to the manufacturer's instructions (Qiagen Inc., Valencia Calif.). Each p53 exon (5-9) was amplified individually from genomic DNA, using the primer sequences and conditions specified.⁸ All cases with identified p53 mutations were repeated with identical results.

### Example 7

### Response of Patients to Alemtuzumab

A total of 36 CLL patients treated with alemtuzumab for whom viably preserved CLL cells were available prior to therapy were studied for the presence of del(17p13.1) or p53 mutations. Of these 36 patients, 15 (42%) had p53 mutations or deletions. These included 8 with both a point mutation and deletion (17p13.1), 4 del(17p13.1), and 3 p53 point mutations The specific details of these mutations are summarized in Table 1. None of the p53 mutations noted in this patient group were silent mutations or known polymorphisms of p53. The clinical characteristics of these 36 patients include a median age of 61 (range 42-74) with 7 (19%) being female. The patients received a median of 3 (range 1-12) therapies, and 29 (81%) were fludarabine refractory. Advanced stage (modified Rai 3 or 4) was present in 27 (75%) of patients. Clinical features among patients with and without p53 mutations or deletions were similar (data not shown).

Of the 36 patients reported herein, 2 (6%) of the patients attained a complete response and 8 (22%) a partial response utilizing the NCI 96 criteria. Response among the 15 patients with p53 mutation and/or deletion was noted in 6 (40%), whereas only 4 (19%) of patients without p53 mutation and/or deletion responded to therapy Among the patients with p53 mutations, the median duration of response was 8 (range 3-17) months. Clinical responses were noted in patients with both presence of mutation and deletion (4 of 8 patients responding) versus those with a deletion or mutation (2 of 7 patients responding).

The data presented herein represent to our knowledge the first series of CLL patients demonstrating that alemtuzumab is effective at eliminating disease that has aberrant p53 function from a mutation and/or gene deletion. This finding is quite relevant to the therapy of CLL given both the high frequency (42%) of p53 dysfunction that we have demonstrated exists in fludarabine-refractory CLL and the inability of other therapies including chlorambucil, fludarabine, and rituximab to work in this setting.¹⁻⁴ A similarly high frequency of p53 mutations has been noted by others^{9,10} in previously treated CLL patients including Sturm and colleagues¹⁰ who noted a 29% frequency in those exposed to prior alkylating therapy as compared to a 5% frequency in previously un-treated patients. Sturm and colleagues¹⁰ and others¹¹⁻¹³ have also demonstrated that significant in vitro resistance to both ex vivo treatment with irradiation, fludarabine, chlorambucil and other alkylator-based therapies is present in the subset of patients with p53 mutations.

TABLE 1
P53 gene Mutations Detected by DGGE and Sequencing

| Patient # | del(17p13.1) | Exon | Sequence Alteration |
|---|---|---|---|
| 1 | yes - 77.0% | 5,8 | TGC > TTT, Cys > Phe, bp13206-7 + |
| | | | CGT > CAT, Arg > His, bp 14487 |
| 2 | yes - 51.5% | 6 | CTT > CGT, Leu > Arg, bp 13341 |
| 3 | yes - 85.5% | 6 | CGA > CAA, Arg > Gln, bp 13398 |
| 4 | yes - 80.5% | 7 | GGC > AGC, Gly > Ser, bp14057 |
| 5 | yes - 61.0% | 7 | AGG > AGT, Arg > Ser, bp 14110; |
| | | | at splice site |
| 6 | yes - 14.5% | 7 | AGG > AAA, Glu > Lys, bp 14099 |
| 7 | no | 7 | CGG > TGG, Arg > Trp, bp 14069 |
| 8 | yes - 97.0% | 7 | GGC > AGC, Gly > Ser, bp14060 |
| 9 | no | 7 | ATG > GTG, Met > Val, bp 14063 |
| 10 | yes - 91.0% | 8 | 26 bp deletion; splice site deleted |
| 11 | no | 8 | GAG > TAG, Glu > Stop, bp 14522 |

The data described support the case report of Stilgenbauer and colleagues⁶ who demonstrated a complete response in a single CLL patient with del(17)(p13.1) and p53 mutation. Similar to the results reported in this single case report, several patients included in our series had durable remissions that ranged from 3 to 17 months. Our findings indicate that alemtuzumab (Campath-1H), as opposed to fludarabine, chorambucil, or rituximab would be a more rational initial treatment choice for patients with p53 mutations and/or del(17)(p13.1), and provide preliminary evidence for screening all patients at time of initial and subsequent therapies for the presence of del(17p13.1) and p53 mutations to avoid administration of otherwise ineffective therapy for this disease.

### REFERENCES

1. Dohner H, Fischer K, Bentz M, Hansen K, Benner A, Cabot G, Diehl D, Schluek R. Coy J, Stilgenbauer S, et al. p53 gene deletion predicts for poor survival and non-response to therapy with purine analogs in chronic B-cell leukemias. Blood. 1995; 85:1580-1589
2. Wattel E, Preudhomme C, Hecquet B, Vanrumbeke M, Quesnel B, Dervite I, Morel P, Fenaux P. p53 mutations are associated with resistance to chemotherapy and short survival in hematologic malignancies. Blood. 1994; 84:3148-3157
3. el Rouby S, Thomas A, Costin D, Rosenberg C R, Potmesil M, Silber R, Newcomb E W. p53 gene mutation in B-cell chronic lymphocytic leukemia is associated with drug resistance and is independent of MDR1/MDR3 gene expression. Blood. 1993; 82:3452-3459
4. Byrd J C, Smith L, Hackbarth M L, Flinn I W, Young D, Proffitt J H, Heerema N A. Interphase cytogenetic abnormalities in chronic lymphocytic leukemia may predict response to rituximab. Cancer Res. 2003; 63:36-38
5. Keating M J, Flinn I, Jain V, Binet J L, Hillmen P, Byrd J, Albitar M, Brettnall L, Santabarbara P, Wacker B, Rai K R. Therapeutic role of alemtuzumab (Campath-1H) in patients who have failed fludarabine: results of a large international study. Blood. 2002; 99:3554-3561
6. Stilgenbauer S, Dohner H. Campath-1H-induced complete remission of chronic lymphocytic leukemia despite p53 gene mutation and resistance to chemotherapy. N Engl J. Med. 2002; 347:452-453
7. Cheson B D, Bennett J M, Grever M, Kay N, Keating M J, O'Brien S, Rai K R. National Cancer Institute-sponsored Working Group guidelines for chronic lymphocytic leukemia: revised guidelines for diagnosis and treatment. Blood. 1996; 87:4990-4997
8. Rines R D, van Orsouw N J, Sigalas I, Li F P, Eng C, Vijg J. Comprehensive mutational scanning of the p53 coding region by two-dimensional gene scanning. Carcinogenesis. 1998; 19:979-984
9. Cordone I, Masi S, Mauro F R, Soddu S, Morsilli 0, Valentini T, Vegna M L, Guglielmi C, Mancini F, Giuliacci S, Sacchi A, Mandelli F, Foa R. p53 expression in B-cell chronic lymphocytic leukemia: a marker of disease progression and poor prognosis. Blood. 1998; 91:4342-4349
10. Sturm I, Bosanquet A G, Hermann S, Guner D, Dorken B, Daniel P T. Mutation of p53 and consecutive selective drug resistance in B-CLL occurs as a consequence of prior DNA-damaging chemotherapy. Cell Death Differ. 2003; 10:477-484
11. Silber R, Degar B, Costin D, Newcomb E W, Mani M, Rosenberg C R, Morse L, Drygas J C, Canellakis Z N, Potmesil M. Chemosensitivity of lymphocytes from patients with B-cell chronic lymphocytic leukemia to chlorambucil, fludarabine, and camptothecin analogs. Blood. 1994; 84:3440-3446
12. Pettitt A R, Sherrington P D, Stewart G, Cawley J C, Taylor A M, Stankovic T. p53 dysfunction in B-cell chronic lymphocytic leukemia: inactivation of ATM as an alternative to TP53 mutation. Blood. 2001; 98:814-822
13. Pepper C, Thomas A, Hoy T, Tighe J, Culligan D, Fegan C, Bentley P. Leukemic and non-leukemic lymphocytes from patients with Li Fraumeni syndrome demonstrate loss of p53 function, Bcl-2 family dysregulation and intrinsic resistance to conventional chemotherapeutic drugs but not flavopiridol. Cell Cycle. 2003; 2:53-58

1. A method for predicting the response of a patient with chronic lymphocytic leukemia to treatment with a therapeutic agent that specifically binds to CD20 antigens on the surface of B-lymphocytes and activates the complement cascade and immune effector cells in the patient, thereby depleting B lymphocytes from the patient's peripheral blood, lymph nodes and hone marrow, comprising:
collecting from the patient a cell sample; and
analyzing the cell sample to detect one or more cytogenetic abnormalities selected from the group consisting of del(17p13.1), del(13q14.3), del(11q22-q23) and trisomy 12;
wherein the presence of the del(17p13.1) abnormality is indicative that the patient will be refractory to treatment with said agent, and wherein the absence of the del(17p13.1) abnormality together with the presence of one or more of the del(13q14.3), del(11q22-q23) and trisomy 12 abnormalities is indicative that the patient will be responsive to treatment with said agent.

2. The method of claim 1 wherein the therapeutic agent is rituximab.

3. The method of claim 1 wherein the cell sample comprises B lymphocytes.

4. The method of claim 1 wherein analysis of the cell sample uses polynucleotide probes and fluorescence in situ hybridization (FISH).

5. The method of claim 4 wherein the FISH uses the LSI p53 probe to detect del(17p13.1).

6. The method of claim 4 wherein the FISH uses the LSI D13S319 probe to detect del(13q14.3).

7. The method of claim 4 wherein the FISH uses the CEP 12 probe to detect trisomy 12.

8. The method of claim 4 wherein the FISH uses the ATM probe to detect del(11q22.3).

9. A diagnostic kit for determining the chemosensitivity of a chronic lymphocytic leukemia patient to treatment with a therapeutic agent that is specific for CD20+ B lymphocytes, comprising:
one or more polynucleotide probes, each of which comprises a polynucleotide sequence which is complementary to and hybridizes under stringent conditions with a target region of chromosomal DNA in a human
wherein one of the probes is complementary to and hybridizes under stringent conditions with the target region 17p13.1 of chromosome 17, and wherein the other probes are complementary to and hybridize under stringent conditions with the target regions selected from the group consisting of the 13q14.3 region of chromosome 13, the 12p11.1-q11 region of chromosome 12, and the 11q22.3 region of chromosome 12.

10. The kit of claim 9 wherein the probes comprise polynucleotides from 50 to 10⁵ nucleotides in length.

11. The kit of claim 10 wherein the probes are selected from the group consisting of oligonucleotides, cDNA molecules, RNA molecules, and synthetic gene probes comprising nucleobases.

12. The kit of claim 9, wherein the probe for the target region 17p13.1 of chromosome 17 is the LSI p53 probe, the probe for the 13q14.3 region of chromosome 13 is the LSI D13S319 probe, the probe for the 12p11.1-q11 region of chromosome 12 is the CEP 12 probe, and the probe for the 11q22.3 region of chromosome 11 is the ATM probe.

13. A method of identifying a patient as likely to be refractory to treatment with rituximab, comprising;
analyzing the genome of cells obtained from the patient for the presence of del(17p13.1), wherein presence of del(17p13.1) indicates that the patient is likely to be refractory to treatment with rituximab.

14. The method of claim 13 wherein analysis of the genome uses fluorescence in situ hybridization (FISH).

15. The method of claim 14 wherein the FISH uses the LSI p53 probe.

16. A method of identifying a patient as likely to respond to treatment with rituximab, comprising;
analyzing the genome of cells obtained from the patient for the presence of del(17p13.1), and one or more of del(13q14.3), del(11q22.3) and trisomy 12,
wherein absence of del(17p13.1) and presence of one or more of del(13q14.3), del(11q22.3) or trisomy 12, indicates the patient is likely to respond to treatment with rituximab.

17. The method of claim 16 wherein analysis of the genome uses fluorescence in situ hybridization (FISH).

18. The method of claim 17 wherein the FISH uses the LSI p53 probe to detect del(17p13.1).

19. The method of claim 17 wherein the FISH uses the LSI D13S319 probe to detect del(13q14.3).

20. The method of claim 17 wherein the FISH uses the CEP 12 probe to detect trisomy 12.

21. The method of claim 17 wherein the FISH uses an approximately 500 kb probe that hybridizes to a locus from D11S1828-D11S1294 to detect del(11q22.3), wherein the probe includes a portion that hybridizes with the Ataxia telangiectasia mutated (“ATM”) gene.

22. The method of claim 21 wherein the 500 kb probe is designated as ATM.

23. A method for predicting the response of a patient with chronic lymphocytic leukemia to treatment with a therapeutic agent that specifically binds to CD52 antigens on the surface of B-lymphocytes and activates the complement cascade and immune effector cells in the patient, thereby depleting B lymphocytes from the patient's peripheral blood, lymph nodes and bone marrow, comprising, comprising:
collecting from the patient a cell sample; and
analyzing the cell sample to detect the cytogenetic abnormality del(17p 13.1),
wherein the presence of the del(17p13.1) abnormality is indicative that the patient will be responsive to treatment with the agent.

24. The method of claim 23 wherein the therapeutic agent is alemtuzumab.

25. The method of claim 23 wherein the cell sample comprises B lymphocytes.

26. The method of claim 23 wherein analysis of the cell sample uses polynucleotide probes and fluorescence in situ hybridization (FISH).

27. The method of claim 26 wherein the FISH uses the LSI p53 probe to detect del(17p 13.1).

28. A method of identifying a patient as likely to respond to treatment with alemtuzumab, comprising;
analyzing the genome of cells obtained from the patient for the presence of del(17p13.1,
wherein the presence of del(17p13.1) indicates the patient is likely to respond to treatment with alemtuzumab.

29. The method of claim 28 wherein analysis of the genome uses fluorescence in situ hybridization (FISH).

30. The method of claim 29 wherein the FISH uses the LSI p53 probe to detect del(17p13.1).
